(19) United States
(12) Patent Application Publication
(10) Pub. No.: US 2005/0257718 A1
(43) Pub. Date: Nov. 24, 2005

(54) Natural Pearl in Butylene Glycol

(75) Inventors: Gabriel E. Uzunian, Rye, NY (US); Leila S. Song, Putnam Valley, NY (US); Dennis F. Turner, Perry, ME (US); Robert A. Lewis, Eastport, ME (US)

Correspondence Address: ENGELHARD CORPORATION, 101 WOOD AVENUE, ISELIN, NJ, 08830, US

(73) Assignee: ENGELHARD CORPORATION, Iselin, NJ (US)

(21) Appl. No.: 10/908,503

(22) Filed: May 14, 2005

Related U.S. Application Data
(60) Provisional application No. 60/573,271, filed on May 22, 2004.

(51) Int. Cl. C04B 2/00; C08K 5/00; C11C 3/00; C09D 11/06; A61K 7/021; A61K 7/025; A61K 7/035

(52) U.S. Cl. 106/504; 424/63; 424/64; 424/69; 106/499; 106/493

(57) ABSTRACT

A natural pearlescent pigment derived from fish is uniformly dispersed in a polybutylene glycol vehicle for use, for example, in cosmetic formulations.

This patent application is a conversion of and claims priority to provisional patent application Ser. 60/573,271 filed May 22, 2004.

### FIELD OF THE INVENTION

The present invention is directed to improved natural pearlescent pigments and, in particular, to an improved dispersion of natural pearlescent pigments.

### BACKGROUND OF THE INVENTION

Laminar or plate-like pigments which impart a pearly or nacreous luster into objects on which or in which they are used are known as “effect” pigments, and have also been known as pearlescent pigments or nacreous pigments. These effect pigments include naturally occurring substances such as pearlescence, a mixture of guanine and hypoxanthine that is obtained from fish.

The manufacture of pearl essence from natural products was primarily a European industry until the First World War when the United States became a significant manufacturer of this product. In broad terms, the natural pearl material was initially obtained from fish scales by agitation or scrubbing with water, optionally warm, or which may have contained ammonia or chemicals which acted as washing compounds. The crystals were isolated and washed. Thereafter, the crystals were thinned with ammonia water and mixed with a lacquer which has a greater affinity for the crystals. The lacquer was isolated and used as a pearlescent paste.

For additional background, one may consult Mattin, Pearl Essence Facts, page 13 (September 1932) and a pamphlet published by Rinshed-Mason Company entitled “Pearl Essence, Historical and Descriptive Data.”

It is a common practice that toiletries, such as shampoos, hair rinses, lotions, creams, soaps, cosmetics, and the like are imparted with pearlescence in order to improve their attractiveness and to enhance their value as commercial products. Hitherto known pearlescent agents used to impart such pearlescence are thin leaf materials of natural origin such as natural crystalline guanine and mica, of which the former is particularly preferred.

Since it is difficult to achieve a high solids content of natural pearl without destroying the crystalline structure, natural pearl pigments have limited industrial applications. However, natural pearl essence has a satiny luster that creates soft, cloud-like mists and deep luster. Many cosmetic and personal care products contain natural pearl pigments to increase luster, depth, iridescence, and pearlescence, and to provide for a soft, shimmering, pearly effect product.

For use in toiletries, the natural pearlescent paste is provided in the form of the natural pearl material dispersed in isopropyl alcohol, among other limited vehicles. Like isopropyl alcohol, some of these vehicles are flammable or are otherwise volatile and must be carefully handled. Recently, butylene glycol (butanediol), a humectant, is replacing the widely used propylene glycol in some personal care products in as much as butylene glycol shows less skin irritation than propylene glycol. Butylene glycol is a colorless and almost odorless liquid. The features provided by this hygroscopic diol include benefits such as solubility in water, alcohols, esters and ketones, glycol ethers, and glycol ether acetates. Further, butylene glycol dispersions have the added benefit of removing the issues of flammability and odor from high VOC-containing dispersions. The industrial hygiene considerations of plant processes are considerably improved when production is achieved using environmentally friendly vehicles such as butylene glycol. The industrial hazardous waste from production is eliminated when compared to the production of products containing VOCs.

Natural pearl has never been dispersed or sold in an alkylene glycol vehicle such as propylene glycol or butylene glycol. The need for natural pearlescent pigments still remains for generating quality color effects in a variety of commercial applications including, for example, cosmetics. In this regard, it is important that the natural pearlescent agents be dispersed in safe, useful and odor free vehicles. Butylene glycol, however, is immiscible in the aliphatic and aromatic hydrocarbons that were used to aid extraction of the pearl crystals from fresh scales and used currently as a coalescing agent when separating crystals from aqueous extraction liquors prior to refining. Accordingly, there is a need to develop a butylene glycol dispersion process for natural pearl.

### SUMMARY OF THE INVENTION

According to the invention, natural pearl is uniformly dispersed into butylene glycol. The resultant dispersed paste can be incorporated into a variety of toiletry products, including cosmetics to impart the desired pearlescent color effect. Nonionic surfactants have been found useful in forming the uniform dispersion of natural pearl into butylene glycol.

### DETAILED DESCRIPTION OF THE INVENTION

The initial steps in providing the novel pearlescent paste of this invention involve separating the natural pearlescent pigment from the fish. These steps are known in the art and do not, per se, form part of the novelty of this invention. The process set forth below represents one way for initially washing and/or treating the crystals for use in a uniformly dispersed paste. Any other method which can yield a clean crystal ready for incorporation into a vehicle that itself can be incorporated into products such as cosmetics can be used.

The first step involves separating the native guanine crystals from the fish such as the scales. This step employs a hot water washing. It has been found that if the water is too cold, only a small portion of the crystals will be removed from the scales and if the water is too hot, it will cause the scales to curl thereby making the crystals largely inaccessible for extraction. It has been found that the water temperature should be about 30° C. to 50° C. and preferably from about 34° C. to 40° C.

A quantity of water sufficient to extract the guanine is combined with the fish scales which may have been previously washed with cold or warm (20° C.) water to remove extraneous matter. Conveniently, the amount of water is in the range of about 1½ to 2½ times the weight of the scales which, in general, have a total solids content of about 30% to 40%. Under these circumstances, adding water having a temperature of about 50° C. results in a final mixing temperature of about 34° C. to 40° C., the range of optimum extraction of the pearl essence crystals.

The mixing of the scales with the hot water is accomplished with agitation. This may be affected using various mixing devices such as troughs with ribbon screws or tanks with propeller agitators. Another alternative is to use a low shear pump such as that used for transferring fruit and vegetables. The duration of the agitated mixing will vary depending on the condition of the scales being treated but, in general, ranges from about 5 to 15 minutes.

The resulting extract liquor is separated from the extracted scales by any suitable means such as a screen or filter. If desired, the separated scales can be rinsed with water, one or more times, at temperatures ranging from ambient to about 50° C. It is preferred to combine and rinse liquor, after separation from the scales, with the initial extract liquor.

The aqueous extract is then concentrated by, for example, gravity settling, centrifugation, or combinations thereof. Using centrifugation, the pearl essence crystals are recovered in the form of an aqueous paste. In one preferred procedure, the combined extract and rinse liquids are permitted to settle for an extended period of time, e.g., overnight, during which time the major amount of the pearl essence crystals accumulate in the lower fraction, which generally comprises 15% to 25% of the total volume. This lower fraction is separated providing a concentrated aqueous slurry. The top portion of the settled extract/rinse water can be centrifuged to recover any pearl essence crystals which may be present and the resulting water can be reused in the first step of the present process. The product is a concentrated aqueous pearl essence slurry or paste which contains a significant quantity of impurities. It is therefore subjected to second stage processing.

In the second stage, pearl essence crystals are preferentially transferred from the concentrated aqueous paste into the organic phase by the method of coalescing. As a result of the transfer, most of the extraneous material is separated from the crude pearl essence concentrate. Organic coalescing agents which can be employed in the process of the present invention include aliphatic and aromatic hydrocarbons, castor oil, soybean oil, jojoba oil, mineral oil, naphtha, isoparaffins, lanolin oil, lard oil, lecithin, organic esters of long chain alcohols such as octyl acetate, and various other vegetable and fish oils as well as mixtures of organic liquids and surfactants. Preferably, the organic contains about 0.1 to 10 wt %, preferably about 4 to 6 wt %, of a nonionic surfactant such as polyoxyethylene sorbitan monooleate, or an alkyl sulfosuccinate such as Aerosol OT, or a fatty acid salt such as sodium oleate, and the like. The organic coalescing agent will generally comprise about 30% to 60% by weight based on the weight of the resultant flushed paste. The combination of the organic coalescing agent and concentrated extract is mixed and sufficient mixing can be determined by observation. Thus, the mixing is deemed to be at an appropriate level when the pearl crystals combine with the organic solvent to form small beads which can be separated from the water phase which contains most of the impurities found in the original concentrate. In general, the mixing is continued for about ¼ of an hour to one hour.

After separating the flushed pearl essence paste from the water by any suitable means, such as a screen or filter, the coalesced paste can be further washed with water, ammonia water, or water containing a small amount of surfactant. The resulting product of the invention is free from most impurities, has good storage characteristics, and is ready to be dispersed into the desired alkylene glycol vehicle.

Natural crystallized guanine, however, may contain impurities, believed to be amines, which can cause deterioration of and as well provide an unpleasant odor in the products formulated therewith. The unpleasant odor may still persist even after several bleaching and washing steps. Accordingly, as a recent trend in the industry of toiletries, natural crystallized guanine is being replaced with synthetic materials which are more readily available and also capable of exhibiting pearlescence. The effect pigments which are most often encountered commercially are titanium dioxide-coated mica and iron oxide-coated mica. Other synthetic effect pigments which have been developed for both cosmetic and industrial use include materials such as bismuth oxychloride and lead carbonate. However, natural pearl essence provides a superior luster and is desired in higher end products.

In commonly assigned, copending application U.S. Ser. No. 10/747,668, filed Dec. 29, 2003, is disclosed a method of deodorizing natural pigments from fish. The deodorization can be carried out in a simple manner by contacting the natural crystallized guanine with a complex hydride, such as a sodium borohydride. Deodorization is achieved without significant crystal degradation and associated loss of luster caused by crystal fragmentation. In this procedure, it is believed the compounds that cause the fish odor are removed from the pearlescent material or otherwise reduced or neutralized. The entire contents of U.S. Ser. No. 10/747,668 are herein incorporated by reference.

As disclosed in the above application, either immediately from the washing step or from storage, the pearlescent pigment paste, which comprises approximately 30-40% of the pearlescent pigment material, may be treated with the complex hydride. As sodium borohydride (NaBH₄) is the preferred complex hydride, the process will be explained with the use of such material. It is to be understood equivalent complex hydrides, including those previously described can be used in place of the sodium borohydride compound. The sodium borohydride can be applied to the pearlescent pigment paste in aqueous solution in concentrations preferably above 10 wt %. However, to avoid immediate and excessive hydrogen off-gassing, the sodium borohydride is preferably added to the pigment paste as a powder. The sodium borohydride powder, for example, can be sprinkled or otherwise applied onto the paste. The amount of the sodium borohydride applied whether in the form of an aqueous solution or solid will range from about 0.5 to about 10% by weight sodium borohydride relative to the pigment paste. More typical amounts of the sodium borohydride added to the pigment paste range from about 0.75-5 wt. % and, more preferably, about 1 wt. % of the sodium borohydride relative to the pearlescent paste is added. The pearlescent pigment paste and sodium borohydride powder are mixed until a uniform mixture is achieved. Any known type of mixing equipment can be used. The pearlescent pigment paste and sodium borohydride are mixed for about two minutes to two hours, more typically for less than one hour and more preferably from about three to ten minutes to form a uniform mixture.

Temperature of treatment and mixing will generally be at ambient conditions. Temperatures up to about 50° C. can be utilized. Upon forming a uniform mixture, sufficient water is added to form a flowable or pumpable paste. Some water may be added to enhance mixing. Typically the amount of water added to the pearlescent pigment paste will range from about 50% to about 200%. More typically, about 100% by weight water relative to the pigment paste is needed to form a flowable or pumpable mixture.

Subsequent to the formation of a pumpable liquid dispersion of pearlescent pigment paste, sodium borohydride, and water, a small amount ranging from about 0.5 to 10 wt. %, preferably about 0.75 to 5 wt %, and, more preferably, about 1 wt %, of a weak acid relative to pearlescent paste is added to the pumpable mixture or slurry. The addition of the weak acid neutralizes both the finished product and the liquid wastewater, providing improved odor reduction. Again, ambient temperature conditions up to 50° C. can be utilized. The process is characterized by foaming and off-gassing from the slurry. This foaming and off-gassing of hydrogen will happen when mixing just the water, sodium borohydride and weak acid without the pearl paste. It is important to add the weak acid soon after the mixture of pearlescent pigment paste, sodium borohydride, and water are provided in a pumpable slurry. Delaying the acid neutralization prevents or reduces the significant off-gassing which appears to be needed to provide successful odor reduction. Accordingly, if the mixture is allowed to sit too long, the addition of the acid does not result in the desired off-gassing or foaming action which is needed. Accordingly, delays of adding the acid once the mixture of pearlescent pigment paste and sodium borohydride is provided should not generally exceed three hours. Once the off-gassing and foaming subsides, additional water can be added to rinse away residual materials. The slurry can then be separated such as by centrifugal action and the pearlescent pigment material which is separated can be dispersed into the appropriate vehicle in which it will eventually be utilized.

While not wishing to be bound by any particular theory, it is believed that the odor contamination in the pearlescent pigment material derived from fish is influenced by amine compounds. The borohydride-acid system which is used to treat the pearlescent pigment paste is thought to affect the odor diminution through a first reduction of odoriferous compounds including lower amines such as dimethyl amine and a second weak Lewis acid-base reaction to release the reduced compounds.

The weak acids which can be added to the slurry of pearlescent pigment paste and borohydride to induce the off-gassing and foaming of the borohydride-treated pearlescent pigment paste are those with relatively low disassociation constants, unlike strong acids such as sulfuric or phosphoric acids. A non-limiting list of useful weak acids includes organic acids such as formic acid, acetic acid, C₃₊ alkanoic acids, citric acid, malic acid, lactic acid, etc. Weak inorganic acids such as nitric and nitrous acid can also be used. Acetic acid is preferred.

The deodorized paste, which typically contains 10-60% of the guanine crystals and the balance water and surfactant, needs to be treated so as to disperse the guanine crystals or the natural pearlescent material into the desired alkylene glycol vehicle. More typically, the deodorized paste will contain 20-40% by weight of guanine crystals, up to about 5% surfactant, with the balance water. The pearlescent pigment paste is mixed, for example, with the butylene glycol vehicle by slowly adding the butylene glycol to the paste in an amount of butylene glycol:paste of about 0.75:1 to about 4:1 by weight, typically in amounts of 1:1 to 3:1. To uniformly disperse the pearlescent pigment material into butylene glycol, it is preferred to add an additional amount of surfactant to that which may be included in the paste from the process of separating the guanine crystals from the fish scales and subsequent cleaning and separating processes. Thus, levels of additional surfactant relative to the paste can be from about 0.5 to about 10% by weight, typically from about 1-10% by weight, and, more preferably, from about 3-7% by weight. While anionic, nonionic, and amphoteric surfactants are believed suitable, it is preferred to utilize nonionic surfactants and, in particular, nonionic surfactants which are in the hydrophilic sector range of the HLB (hydrophilic-lipophilic balance) scale. Thus, nonionic surfactants having an HLB of at least 12 are useful. Surfactants with HLB values of at least 14.5 are particularly useful. Among the most useful surfactants are the polyoxyethylene derivatives of sorbitan fatty acid esters. These nonionic surfactants are derived from the partial esters of common fatty acids such as lauric, palmitic, stearic, and oleic acids and the hexitol anhydrides derived from sorbitol. Such surfactants are marketed under the tradename Tween®. Thus, particularly preferred are Tween 20®, Tween 40®, Tween 60®, and Tween 80®, each of which contains 20 oxyethylene units.

Once the pearlescent pigment paste, preferably a pigment paste which has been deodorized by the process described above, is mixed with the butylene glycol and additional surfactant, the slurry is heated to reduce the water content. Thus, the slurry can be heated to a temperature of about 80° C. and maintained at that temperature for a time sufficient to reduce the water content to the desired amount. Thus, holding the temperature at 80° C. for 8 hours can reduce the water content to below 5%. Subsequent to water removal, the paste typically will contain the butylene glycol and from about 10-40% of the guanine crystals. Further dilution with butylene glycol can reduce the pearlescent material content if desired. Typically, a desired level of natural pearlescent pigment within the butylene glycol will range between 10-30%, and most typically around 20 wt. % for incorporation into products for pigmentation.

Products of this invention have use in all types of automotive paint applications. For example, these effect pigments can be used in mass tone or as styling agents to spray paint all types of automotive and non-automotive vehicles. Similarly, they can be used on all clay/formica/wood/glass/metal/enamel/ceramic and non-porous or porous surfaces. The effect pigments can be used in coating compositions or incorporated into plastic articles geared for the toy industry or the home. These effect pigments can be impregnated into fibers to impart new and esthetic coloring to clothes and carpeting. They can be used to improve the look of shoes, rubber and vinyl/marble flooring, vinyl siding, and all other vinyl products. In addition, these colors can be used in all types of modeling hobbies. Natural Pearl Pigments have limited industrial applications, again due to temperature, pH, shear, cost and an inability to achieve high total solids content without destroying crystalline structure.

The above-mentioned compositions in which the compositions of this invention are useful are well known to those of ordinary skill in the art. Examples include printing inks, nail enamels, lacquers, thermoplastic and thermosetting materials, natural resins, and synthetic resins. Some non-limiting examples include polystyrene and its mixed polymers, polyolefins, in particular, polyethylene and polypropylene, polyacrylic compounds, polyvinyl compounds, for example polyvinyl chloride and polyvinyl acetate, polyesters and rubber, and also filaments made of viscose and cellulose ethers, cellulose esters, polyamides, polyurethanes, polyesters, for example polyglycol terephthalates, and polyacrylonitrile. For a well-rounded introduction to a variety of pigment applications, see Temple C. Patton, editor, The Pigment Handbook, volume II, Applications and Markets, John Wily and Sons, New York (1973). In addition, see for example, with regard to ink: R. H. Leach, editor, The Printing Ink Manual, Fourth Edition, Van Nostrand Reinhold (International) Co. Ltd., London (1988), particularly pages 282-591; with regard to paints: C. H. Hare, Protective Coatings, Technology Publishing Co., Pittsburgh (1994), particularly pages 63-288. The foregoing references are hereby incorporated by reference herein for their teachings of ink, paint, and plastic compositions, formulations and vehicles in which the compositions of this invention may be used including amounts of colorants.

In the cosmetic field, the effect materials can be used in all cosmetic and personal care applications subject, of course, to all regulatory requirements. Thus, they can be used in hair sprays, leg-makeup, insect repellant lotion, mascara cake/cream, nail enamel, nail enamel remover, perfume lotion, and shampoos of all types (gel or liquid). In addition, they can be used in shaving cream (concentrate for aerosol, brushless, lathering), skin glosser stick, skin makeup, hair groom, eye shadow (liquid, pomade, stick, pressed, or cream), eye liner, cologne stick, cologne, cologne emollient, bubble bath, body lotion (moisturizing, cleansing, analgesic, astringent), after shave lotion, after bath milk, and sunscreen lotion.

For a review of cosmetic applications, see Cosmetics: Science and Technology, 2^{nd} Ed., Eds: M. S. Balsam and Edward Sagarin, Wiley-Interscience (1972) and deNavarre, The Chemistry and Science of Cosmetics, 2^{nd} Ed., Vols 1 and 2 (1962), Van Nostrand Co Inc., Vols 3 and 4 (1975), Continental Press, both of which are hereby incorporated by reference.

### EXAMPLE

100 grams of a deodorized natural pearlescent pigment paste containing 35.7 wt. % guanine crystals, 62.8 wt. % water, and 1.5 wt. % others such as residual surfactants and hydrocarbons was mixed with 450 grams butylene glycol, 5 grams Tween 80®, and 50 grams water. Mixing was complete when a pumpable slurry in the form of a smooth paste was produced.

The slurry as heated at 80° C. for 8 hours in a steam-jacketed kettle fitted with a mixer to evaporate the water. The total water content after heating was 5 grams determined by Karl Fischer analysis.

The low water content slurry was then centrifuged under 2,000 Gs for 30 minutes to reduce the remaining water and surfactant and concentrate the pearlescent content. 119 grams of a paste containing 30 wt. % pearlescent pigment in butylene glycol was formed. This material was then diluted with supernate from the centrifuged slurry to provide a paste containing 20 wt. % pearlescent pigment.

1. A pigment composition comprising a pigment derived from fish uniformly dispersed within butylene glycol or propylene glycol.

2. The composition of claim 1 wherein said pigment comprises guanine crystals.

3. The composition of claim 1 comprising 10-40% by weight of said pigment.

4. The composition of claim 1 comprising 10-30% by weight of said pigment.

5. The composition of claim 4 comprising 20% by weight of said pigment.

6. The composition of claim 1 further containing a surfactant.

7. The composition of claim 6 wherein said surfactant is a nonionic surfactant.

8. The composition of claim 7 wherein said surfactant has an HLB value of at least 12.

9. The composition of claim 8 wherein said surfactant has an HLB value of at least 14.5.

10. The composition of claim 7 wherein said nonionic surfactant comprises a polyoxyethylene derivative of sorbitan fatty esters.

11. The composition of claim 10 wherein said surfactant comprises 20 oxyethylene units.

12. The composition of claim 1 wherein said pigment has been deodorized by contact with a complex metal hydride.

13. The composition of claim 12 wherein said pigment has been deodorized by contact with sodium borohydride.

14. The composition of claim 1 containing less than 5 wt. % water.

15. The composition of claim 6 containing 0.5 to about 10% by weight surfactant relative to the composition.

16. The composition of claim 15 wherein said surfactant comprises 1-10% by weight of the composition.

17. The composition of claim 11 wherein said surfactant comprises 1-10% by weight of the composition.

18. A cosmetic formulation containing the pigment composition of claim 1.

19. A cosmetic formulation containing the pigment composition of claim 7.

20. A cosmetic formulation containing the pigment composition of claim 17.
